(19) United States
(12) Patent Application Publication
(10) Pub. No.: US 2024/0156525 A1
(43) Pub. Date: May 16, 2024

(54) MEDICAL SYSTEMS FOR ABLATION OR ELECTROPORATION INCLUDING AN EXPANDABLE ELECTRICALLY CONDUCTIVE STYLET AND METHODS OF USE

(71) Applicant: INTUITIVE SURGICAL OPERATIONS, INC., Sunnyvale, CA (US)

(72) Inventors: Serena H. Wong, Los Altos, CA (US); Samuel Raybin, San Jose, CA (US); Randall L. Schlesinger, San Mateo, CA (US)

(21) Appl. No.: 18/510,193

(22) Filed: Nov. 15, 2023

Related U.S. Application Data
(60) Provisional application No. 63/425,973, filed on Nov. 16, 2022.

(51) Int. Cl. A61B 18/14

(52) U.S. Cl. CPC A61B 18/1492; A61B 2018/00577

(57) ABSTRACT

A system may comprise an elongated tool through which a lumen extends and may comprise a stylet including an expandable electrode portion. The expandable electrode portion may have a collapsed configuration within the lumen and may have an expanded configuration outside of the lumen. A diameter of the expandable electrode portion may be larger in the expanded configuration than the collapsed configuration. In the expanded configuration, the expandable electrode portion may be configured to create a plurality of path segments in a target tissue. Each path segment in the plurality of path segments may be extended in a different direction. The expandable electrode portion in the expanded configuration may be configured to deliver energy to ablate the target tissue.

### RELATED APPLICATIONS

This patent claims priority to and benefit of U.S. Provisional Application No. 63/425,973, filed Nov. 16, 2022 and entitled “MEDICAL SYSTEMS FOR ABLATION OR ELECTROPORATION INCLUDING AN EXPANDABLE ELECTRICALLY CONDUCTIVE STYLET AND METHODS OF USE,” which is incorporated by reference herein in its entirety. This patent application is also related to U.S. Provisional Patent Application 63/425,879, entitled “MEDICAL SYSTEMS FOR ABLATION OR ELECTROPORATION INCLUDING A REMOVABLE ELECTRICALLY CONDUCTIVE STYLET AND METHODS OF USE,” filed Nov. 16, 2022, which is incorporated by reference herein in its entirety.

### FIELD

Examples described herein relate to medical systems for energized treatment, such as ablation or electroporation, of target tissue using an elongated tool in which an electrically conductive stylet may be inserted. The stylet may be expandable distally of the elongated tool to generate a plurality of path segments in the target tissue.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools may include instruments such as ablation, electroporation, or other energy delivery instruments. Improved systems and methods are needed to deliver energy to treat large target tissue areas with fewer deployments of the minimally invasive tool.

### SUMMARY

The following presents a simplified summary of various examples described herein and is not intended to identify key or critical elements or to delineate the scope of the claims.

Consistent with some examples, a system may comprise an elongated tool through which a lumen extends and may comprise a stylet including an expandable electrode portion. The expandable electrode portion may have a collapsed configuration within the lumen and may have an expanded configuration outside of the lumen. A diameter of the expandable electrode portion may be larger in the expanded configuration than the collapsed configuration. In the expanded configuration, the expandable electrode portion may be configured to create a plurality of path segments in a target tissue. Each path segment in the plurality of path segments may be extended in a different direction. The expandable electrode portion in the expanded configuration may be configured to deliver energy to ablate the target tissue.

Consistent with other examples, a method may comprise extending an elongated tool into a patient anatomy. The elongated tool may include a lumen extending therethrough. The method may also comprise extending a stylet within the lumen. The stylet may include an expandable portion having a collapsed configuration within the lumen and having an expanded configuration outside of the lumen. A diameter of the expandable portion may be larger in the expanded configuration than the collapsed configuration. The method may also include extending the expandable portion of the stylet distally of the lumen and into a target tissue and expanding the expandable portion to the expanded configuration to generate a plurality of path segments in the target tissue. Each path segment of the plurality of path segments may extend in a different direction from the other path segments. The method may also include applying an electrical current to the stylet to deliver energy to the target tissue through the expandable portion in the expanded configuration.

Other examples include corresponding computer systems, apparatus, and computer programs recorded on one or more computer storage devices, each configured to perform the actions of any one or more methods described below.

It is to be understood that both the foregoing general description and the following detailed description are illustrative and explanatory in nature and are intended to provide an understanding of the various examples described herein without limiting the scope of the various examples described herein. In that regard, additional aspects, features, and advantages of the various examples described herein will be apparent to one skilled in the art from the following detailed description.

### BRIEF DESCRIPTIONS OF THE DRAWINGS

FIG. 1 illustrates a medical instrument system including a delivery device and an elongated tool in which extends an electrically conductive stylet, according to some examples.

FIG. 2A is a cross-sectional side view of an elongated tool in which extends an electrically conductive stylet including a helical portion, according to some examples.

FIG. 2B illustrates the cross-sectional side view of FIG. 2A with the helical portion extended from the elongated tool, according to some examples.

FIG. 3A is a cross-sectional side view of an elongated tool and an electrically conductive stylet in a low-profile configuration within the elongated tool, according to some examples.

FIG. 3B is a cross-sectional side view of an elongated tool and an electrically conductive stylet in an expanded configuration, according to some examples.

FIG. 3C is a cross-sectional side view of an elongated tool and an electrically conductive stylet in an expanded configuration, according to some examples.

FIG. 3D is a cross-sectional side view of an elongated tool and an electrically conductive stylet in an expanded configuration, according to some examples.

FIG. 4 is a cross-sectional side view of an elongated tool and an electrically conductive stylet in an expanded configuration, according to some examples.

FIG. 5A is a cross-sectional side view of an elongated tool and an electrically conductive stylet in a low-profile configuration within the elongated tool, according to some examples.

FIG. 5B is a cross-sectional side view of an elongated tool and an electrically conductive stylet in an expanded configuration, according to some examples.

FIG. 5C is a cross-sectional side view of an elongated tool and an electrically conductive stylet in an expanded configuration, according to some examples.

FIG. 6A is a cross-sectional side view of an elongated tool and an electrically conductive stylet in a low-profile configuration within the elongated tool, according to some examples.

FIG. 6B is a cross-sectional side view of an elongated tool and an electrically conductive stylet in an expanded configuration, according to some examples.

FIG. 7A is a cross-sectional side view of an elongated tool with an expandable portion in a low-profile configuration, according to some examples.

FIG. 7B is a cross-sectional side view of an elongated tool with an expandable portion in an expanded configuration, according to some examples.

FIG. 8A is a cross-sectional side view of a bi-polar medical assembly according to some examples.

FIG. 8B is a cross-sectional side view of a bi-polar medical assembly according to some examples.

FIG. 9 is a flowchart illustrating a method of delivering energy to a target tissue.

FIG. 10 is a simplified diagram of a robot-assisted medical system according to some examples.

FIG. 11A and 11B are simplified diagrams of a medical instrument system according to some examples.

Various examples described herein and their advantages are described in the detailed description that follows. It should be appreciated that like reference numerals are used to identify like elements illustrated in one or more of the figures for purposes of illustrating but not limiting the various examples described herein.

### DETAILED DESCRIPTION

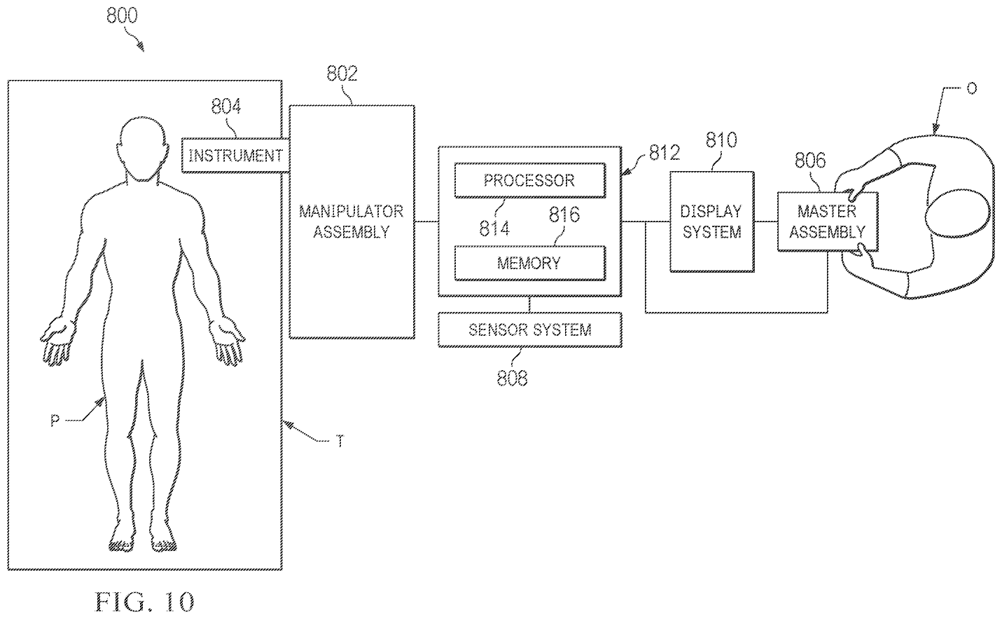

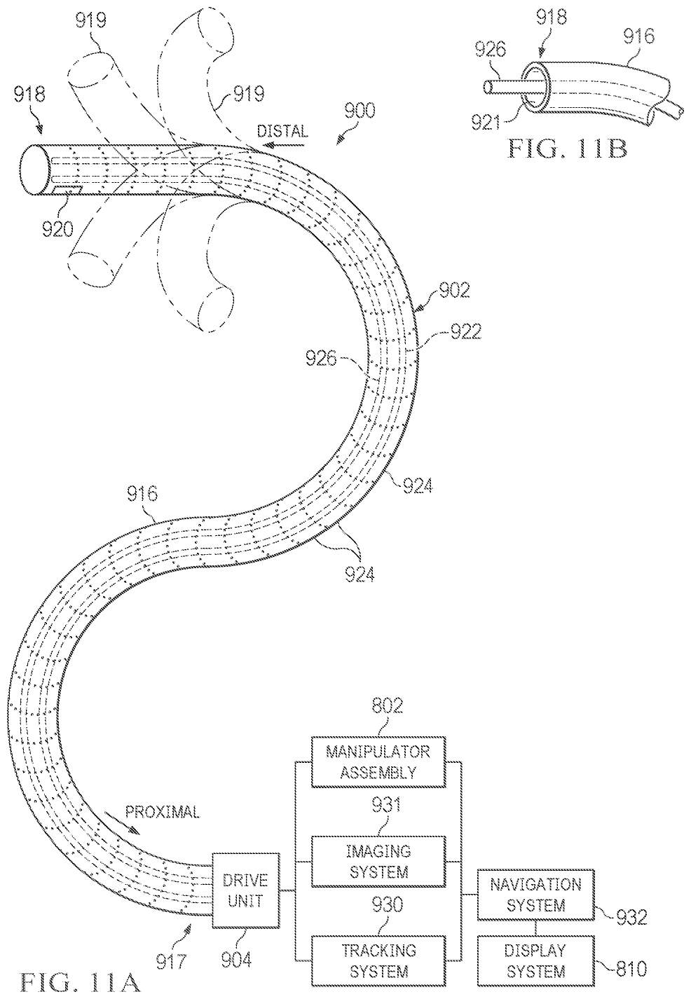

In various examples, an elongated tool, such as a needle, may include a lumen through which one or a series of implements may be passed to conduct therapeutic, diagnostic, or other medical procedures. For example, a biopsy stylet may be inserted through a needle positioned within a target tissue to obtain a tissue sample. The biopsy stylet may be withdrawn and replaced by an electrically conductive stylet that extends distally from the needle and expands an electrically conductive portion to extend into a wider area than the needle itself, to deliver energy to an expanded area of the target tissue. Examples of electrically conductive stylets with various expandable configurations are provided. The systems described herein may be used to perform an energized procedure, such as an ablation or electroporation procedure, on the target tissue. An ablation procedure may deliver heat or cold energy to the target tissue, using for example radio frequency (RF) ablation or cryoablation, to burn, scar, or otherwise destroy localized tissue. For example, RF ablation may be performed using a constant current to generate thermal effects. An electroporation procedure may use high voltage pulses to create temporary pores in cell membranes through which DNA, a drug, or other substance may be introduced into cells. The pores may be created by destroying or modifying cell walls. The present disclosure describes elongated tools that may be used, for example, in medical systems to provide ablation, electroporation, or other treatments that involve the delivery of energy to target tissue. Examples of medical systems that may incorporate any of the flexible elongate devices described herein are provided at FIGS. 10, 11A, and 11B.

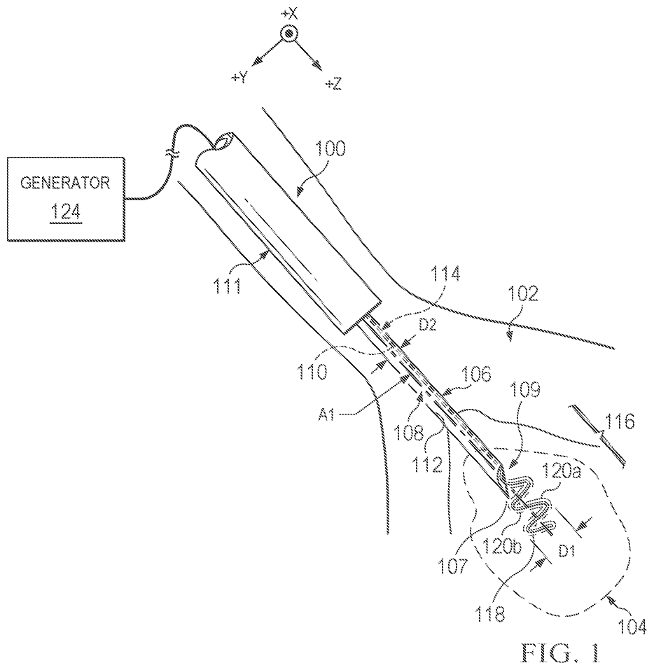

FIG. 1 illustrates a medical instrument system 100 extending with an anatomic passageway 102 and into a target tissue 104. The target tissue may be, for example a tumor, a lymph node, or other tissue to be investigated and/or treated. The medical instrument system 100 may include an elongated tool 106 having a lumen 108 in which extends an electrically conductive stylet 110. Optionally, the medical instrument system 100 may be extended from a delivery device 111, such as a catheter, bronchoscope, or other type of delivery device, which may be navigated within the anatomic passageway 102 and parked near the target tissue to create a deployment location for the elongated tool 106. In some examples, the elongated tool 106 may be flexible with an inner wall 112 defining the lumen 108. The elongated tool 106 may include a pointed tip 107 and an aperture 109 in communication with the lumen 108. In some examples, the tool 106 may be a cannulated needle. The stylet 110 may be flexible and may include a shaft portion 114 and an expandable electrode portion 116 to transmit electromagnetic energy from the stylet 110 to the target tissue 104. When the expandable electrode portion 116 is extended distally of the aperture 109, it may expand to a width dimension D1 that is larger than a width dimension D2 of the tool 106. Distal of the aperture 109, the expandable electrode portion 116 may tunnel through the target tissue 104 to generate a path 118 or tortuous passage within the target tissue 104. A “path,” as used herein, may refer a tunnel, channel, bore, passage, puncture or other cut in the target tissue. A path may include continuous path segments or may include a set of discontinuous path segments. A “path segment,” as used herein, refers to a portion of a path. The path segments of a path may be continuous or discontinuous. The expandable electrode portion 116 may create path segments that extend in different directions. The path segments extending in different directions may be segments of a continuous path or discontinuous path. For example, the path 118 may include path segments 120a, 120b that extend away from a central axis A1 of the lumen 108. The path segments 120a, 120b may extend in different directions relative to the axis A1. In this example, the path segment 120a may extend in a generally −Y direction and the path segment 120b may extend in a generally +Y direction. The path segments may also or alternatively extend in various +/−X directions and +/−Z directions, depending on the three-dimensional shape of the expandable portion 116. Energy may be delivered though the expandable electrode portion to the tissue surrounding the path 118. The expandable electrode portion 116 may allow the stylet to access a larger zone and deliver energy to a larger region of the target tissue 104 (e.g., than a probe alone could access in a single deployment). A target tissue region that would require multiple treatments or deployments into different regions of the target tissue may be accessed with fewer treatments or deployments with the expandable portion. Thus, the expandable electrode portion 116 may generate a larger treatment (e.g., ablation or electroporation) zone than would a single deployment of a straight electrode. The resulting enlarged treatment zone may allow improved access to margin areas of a tumor, may require the patient to undergo fewer treatments, and may decrease the time required to perform a full treatment of the target tissue. The tunneling or boring action of the expandable portion as it extends into the target tissue may prevent cavitation or enlarged void creation in the target tissue.

The stylet 110 and the elongated tool 106 may be formed of any of a variety of electrically conductive materials including stainless steel, titanium, titanium coated stainless steel, or a nickel-titanium alloy (e.g., nitinol). The expandable electrode portion 116 may be formed, for example, from an electrically conductive shape-memory material such as nitinol that may be pre-set to a known shape. In some examples, a plating material may be applied to the expandable electrode portion to provide electrical conductivity. For example, a base material such as stainless steel or nitinol that may have the desired mechanical properties (e.g., durability, strength, elasticity) may be plated with a material such as gold that has superior electrical properties to the base material.

Optionally, the electrically conductive stylet 110 may be coupled to an energy generator 124. The energy generator may be, for example an RF generator or a pressurized gas cryoablation generator for generating heat or cold energy. The energy generator 124 may include components, including hardware, software, and consumable materials, to be used to conduct a variety of ablation or electroporation procedures including pulsed radiofrequency ablation, continuous radiofrequency ablation, water-cooled radio frequency ablation, cryo-neurolysis, cryoablation, microwave ablation, laser ablation, ultrasound ablation, irreversible electroporation, reversible electroporation, or other types of ablation or electroporation. In some examples, the stylet 110 may be removable, freeing the lumen 108 to be used for passage of other tools or substances. For example, a stylet that facilitates biopsy or medications may be passed through the lumen 108 while the stylet 110 is removed. In some examples, the stylet 110 may be used to facilitate biopsy. For example, the presence of the stylet 110 at the needle tip of the tool 106 while it is penetrating tissue protects the needle tip from damage. The stylet 110 may be retracted proximally from the needle tip to collect tissue samples within the lumen 108 of the tool 106.

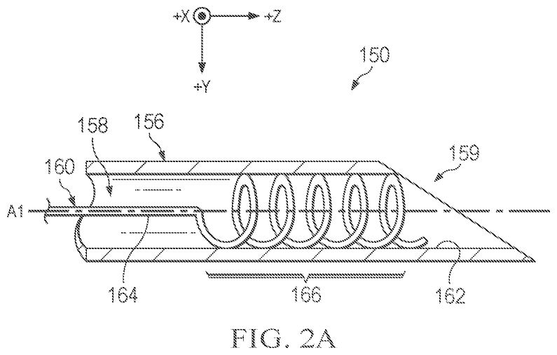

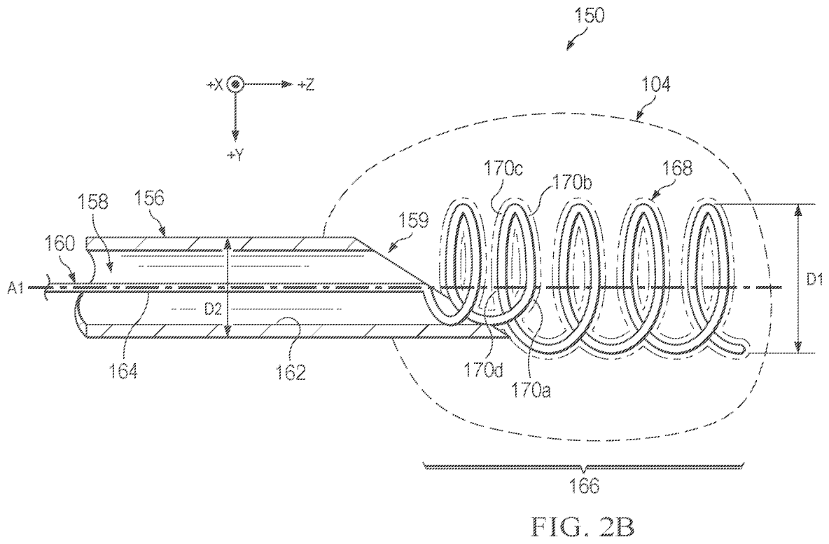

FIGS. 2A and 2B illustrate a cross-sectional side view of a medical instrument system 150 including an elongated tool 156 (e.g., the elongated tool 106) in which extends an electrically conductive stylet 160 (e.g., the stylet 110) including a shaft portion 164 and an expandable electrode portion 166 coupled to or integral with the shaft portion 164. In some examples, the expandable portion 166 may have a helical or twisted shape. In some examples, the shaft portion 164 may be omitted, and the entire length of the stylet may be an expandable portion. The elongated tool 156 may be flexible and may include an inner wall 162 defining a lumen 158. The lumen 158 may extend to a distal aperture 159. An interior diameter or dimension of the tool 156 may have the width dimension D2. As shown in FIG. 2A, when the expandable electrode portion 166 is within the lumen 158, the expandable electrode portion 166 may be compressed, with a width approximately the same dimension D2 as the interior dimension of the constraining inner wall 162.

As shown in FIG. 2B, as the expandable portion 166 is extended distally of the aperture 159 and freed of the constraint of the inner wall 162, it may expand to a width dimension D1 that is larger than the width dimension D2 of the tool 156. Distal of the aperture 159, the expandable portion 166 may tunnel (e.g., with a corkscrew action) through the target tissue 104 to generate a helical or corkscrew shaped path or passage 168 within the target tissue 104. The path 168 may be a continuous path that includes serially connected path segments 170a, 170b, 170c that extend in various different directions relative to the central axis A1 of the lumen 158. For example, the path segment 170a may extend in a generally −Y, +Z direction, the path segment 170b may extend in a generally −Y, −Z direction, the path segment 170c may extend in a generally +Y, −Z, and the path segment 170d may extend in a generally +Y, +Z direction. The path segments may also extend in various +/−X directions. Electricity may be delivered though the expandable electrode portion 166 to the target tissue 104 surrounding the path 168. The expandable electrode portion 166 may allow the stylet to access a larger zone and deliver electricity to a larger region of the target tissue 104 than the tool 156 alone could access in a single deployment.

The stylet 160 and the elongated tool 156 may be formed of any of a variety of electrically conductive materials including stainless steel, titanium, titanium coated stainless steel, or a nickel-titanium alloy (e.g., nitinol) or a combination of materials, including electrically conductive plating materials. The expandable electrode portion 166 may have a helix or otherwise spiral shape and may be referred to as a pigtail, corkscrew, coil-spring or other similar shape. In some examples the shape of the helical portion may be thermally responsive. For example, the portion 166 may be formed of a nitinol material that is preset to assume the helical shape in response to heat energy. In such an example, the nitinol portion may have a non-helical shape in the absence of an applied electrical current, but when an electrical current is applied, the heat energy may cause a modification of the shape and induce the formation of the predetermined helical configuration.

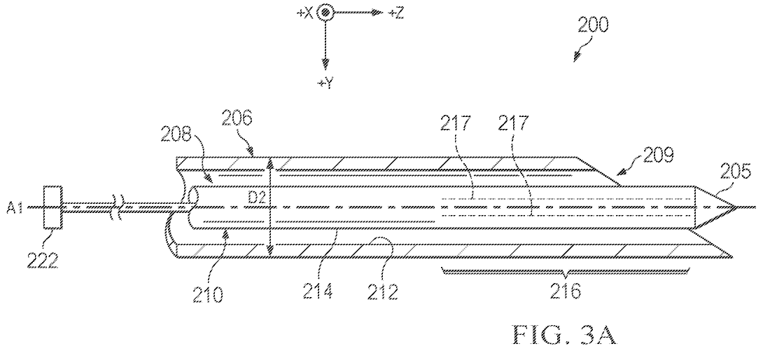

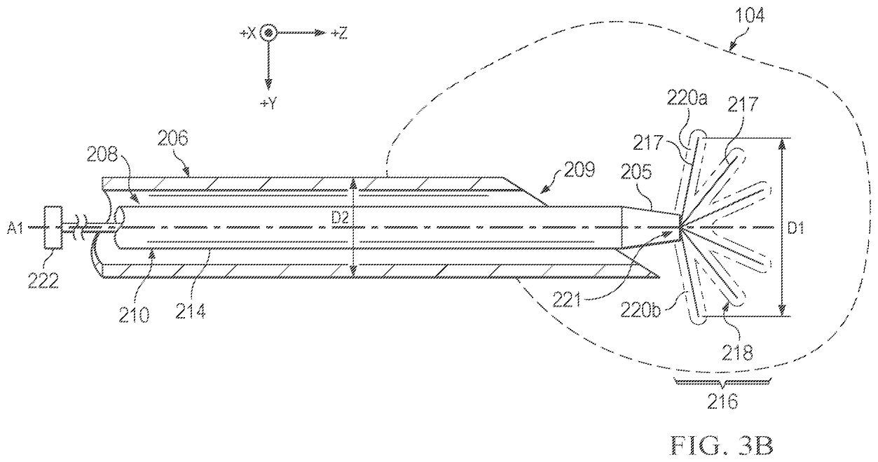

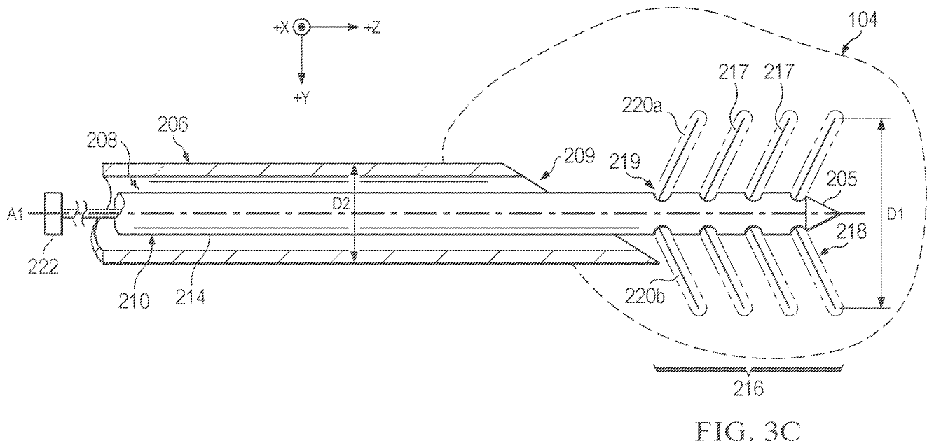

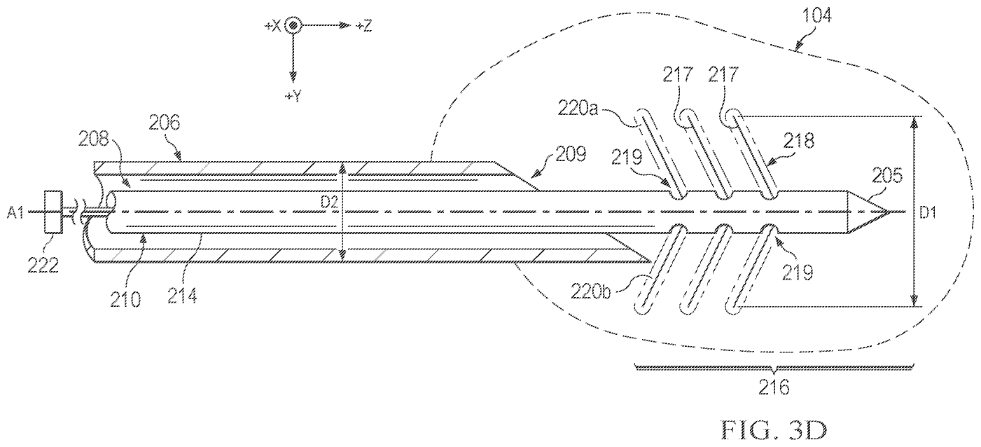

FIGS. 3A-3D illustrate a cross-sectional side view of a medical instrument system 200 including an elongated tool 206 (e.g., the elongated tool 106) in which extends an electrically conductive stylet 210 (e.g., the stylet 110) including a shaft portion 214 and an expandable electrode portion 216. The elongated tool 206 may be flexible and may include an inner wall 212 defining a lumen 208. The lumen 208 may extend to a distal aperture 209. An interior diameter or dimension of the tool 206 may have the width dimension D2. In some examples, the shaft portion 214 may include a tapered distal end 205 that allows the stylet 210 to gain access to the target tissue 104. In this example, the expandable electrode portion 216 may include a plurality of electrode tines 217, and the shaft portion 214 may be cannulated, with the electrode tines 217 compressed and recessed within the shaft portion 214, as shown in FIG. 3A. As shown in the examples of FIGS. 3B, 3C, and 3D, after the shaft portion 214 is extended distally of the aperture 209 and into the target tissue 104, the electrode tines 217 may be extended to expand to a width dimension D1 that is larger than the width dimension D2 of the tool 206. In the examples of FIGS. 3B, 3C, and 3D the electrode tines 217 may each be generally straight and may flare out from the shaft portion 214 and into the target tissue 104 to create path 218. The electrode tines 217 may each tunnel (e.g., create generally linear routes) through the target tissue 104 to create the path. The electrode tines 217 may have a preset shape memory or may be otherwise pre-bent such that they flare outward from the axis A1 when released from the confines of the shaft portion 214.

In the example of FIG. 3B, the electrode tines 217 may extend from an aperture or opening 221 at the distal end 205 of the shaft portion 214 and may flare out radially and/or distally from the axis A1 and into the target tissue 104 to create the path 218. In this example, the tines may extend in a generally +or −Y direction and +Z direction. The tines may also extend in various +/−X directions. The tines may, for example, form a radial or sunburst pattern.

In the example of FIG. 3C, the electrode tines 217 may extend from apertures or openings 219 along a length of the shaft portion 214 and may flare outward radially and/or distally from the axis A1 and into the target tissue 104 to create the path 218. In this example, the tines may extend in a generally + or −Y direction and +Z direction. The tines may also extend in various +/−X directions.

In the example of FIG. 3D, the electrode tines 217 may extend from apertures or openings 219 along a length of the shaft portion 214 and may flare outward radially and/or proximally from the axis A1 and into the target tissue 104 to generate the path 218. In this example, the tines may extend in a generally + or −Y direction and −Z direction. The −Z direction (e.g., generally proximal) of the tines may be useful in preventing the tines from extending distally of the target tissue and into sensitive tissue distal of the target tissue. The tines and resulting path segments may also extend in various +/−X directions.

The path 218 created by the tines 217 may include discrete or discontinuous path segments that extend away from the central axis A1. The path segments may include path segments 220a, 220b. The path segments 220a, 220b may extend in different directions relative to the axis A1. For the examples of FIG. 3B and 3C, the path segment 220a may extend in a generally −Y, +Z direction and the path segment 220b may extend in a generally +Y, +Z direction. For the example of FIG. 3D, the path segment 220a may extend in a generally, −Y, −Z direction and the path segment 220b may extend in a generally +Y, −Z direction. Electricity may be delivered though the expandable electrode portion 216 to the target tissue 104 surrounding the path 218. The expandable electrode portion 216 may allow the stylet to access a larger zone and deliver electricity to a larger region of the target tissue 104 than the tool 206 alone could access in a single deployment.

In some examples, an actuator 222 may be coupled to the expandable electrode portion 216 to cause relative motion between the expandable electrode portion 216 and the shaft portion 214. In FIGS. 3B and 3C, for example, the actuator 222 may be coupled to the expandable electrode portion 216 such that as the actuator 222 is moved (e.g., displaced longitudinally or rotated), the expandable electrode portion 216 may advance distally relative to the shaft portion 214. The distal advancement of the expandable electrode portion 216 may cause the tines 217 to extend distally of the distal end 205 (as in FIG. 3B) or to extend through the apertures 219 (as in FIG. 3C). In FIG. 3D, for example, the actuator 222 may be coupled to the expandable electrode portion 216 such that as the actuator 222 is moved (e.g., displaced longitudinally or rotated), the expandable electrode portion 216 may move proximally relative to the shaft portion 214. The proximal motion of the expandable electrode portion 216 may cause the tines 217 to extend through the apertures 219.

In the various examples of FIGS. 3B-3D, the tines 217 may have a pre-set shape memory or may be otherwise pre-bent such that they flare away from the axis A1 when extended from the shaft portion 214. A reverse motion of the actuator 222 may cause the tines 217 to retract into the shaft portion 214.

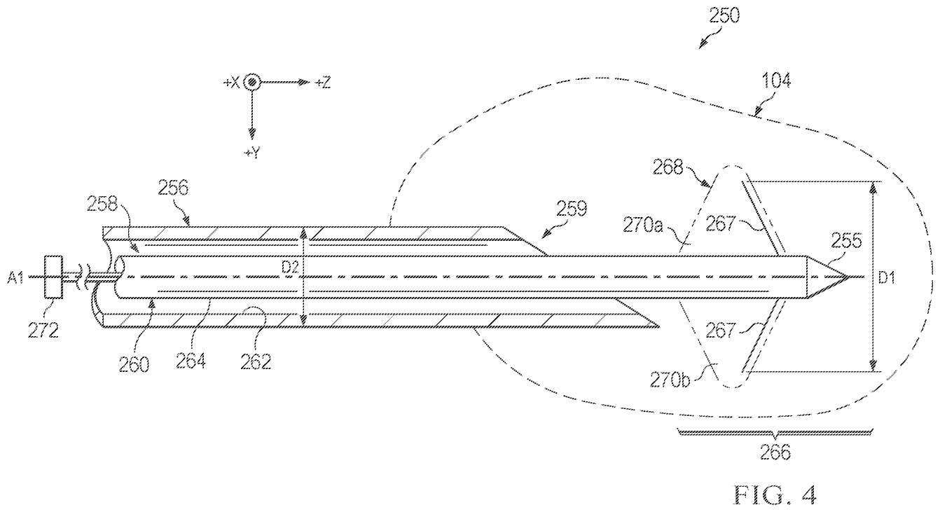

FIG. 4 illustrates a cross-sectional side view of a medical instrument system 250 including an elongated tool 256 (e.g., the elongated tool 106) in which extends an electrically conductive stylet 260 (e.g., the stylet 110) including a shaft portion 264 and an expandable electrode portion 266. The elongated tool 256 may be flexible and may include an inner wall 262 defining a lumen 258. The lumen 258 may extend to a distal aperture 259. An interior diameter or dimension of the tool 206 may have the width dimension D2. In some examples, the shaft portion may include a tapered distal end 255 that allows the stylet 210 to pierce or gain access to the target tissue 104. In this example, the expandable electrode portion 266 may include a plurality of electrode tines 267 that may be compressed against the shaft portion 264 when the expandable electrode portion 266 is within the lumen 258. After the shaft portion 264 is extended distally of the aperture 259 and into the target tissue 104, the electrode tines 267 may be extended to expand to a width dimension D1 that is larger than the width dimension D2 of the tool 256. The electrode tines 267 may each be generally straight and may flare out from the shaft portion 264 and into the target tissue 104 to create path 268. The electrode tines 267 may each fan or slice through the target tissue 104, creating generally wing-shaped planar routes. The electrode tines 267 may have a preset shape memory or may be otherwise pre-bent such that they flare outward from the axis A1 when released from the confines of the shaft portion 264. The electrode tines 267 may flare out radially from the axis A1 and into the target tissue 104. In this example, the tines may extend in a generally + or −Y direction and −Z direction. The tines may also extend in various +/−X directions. The path 268 created by the tines 267 may include discrete or discontinuous planar path segments 270a, 270b that extend away from the central axis A1. The path segments 270a, 270b may extend in different directions relative to the axis A1. For the path segment 270a may extend in a generally −Y direction and the path segment 270b may extend in a generally +Y direction. Electricity may be delivered though the expandable electrode portion 266 to the target tissue 104 surrounding the path 268. The expandable electrode portion 266 may allow the stylet to access a larger zone and deliver electricity to a larger region of the target tissue 104 than the tool 256 alone could access in a single deployment.

In some examples, the tines 267 may have a pre-set shape memory or may be otherwise pre-bent such that they flare away from the axis A1 when extended from the tool 256. In some examples, an actuator 272 may be coupled to the expandable electrode portion 266 to cause relative motion between the expandable electrode portion 266 and the shaft portion 264. The actuator 272 may be coupled to the expandable electrode portion 266 such that as the actuator 272 is moved (e.g., displaced longitudinally or rotated), the tines 267 may fan out relative to the shaft portion 264. A reverse motion of the actuator 272 may cause the tines 267 to move back against the shaft portion 264.

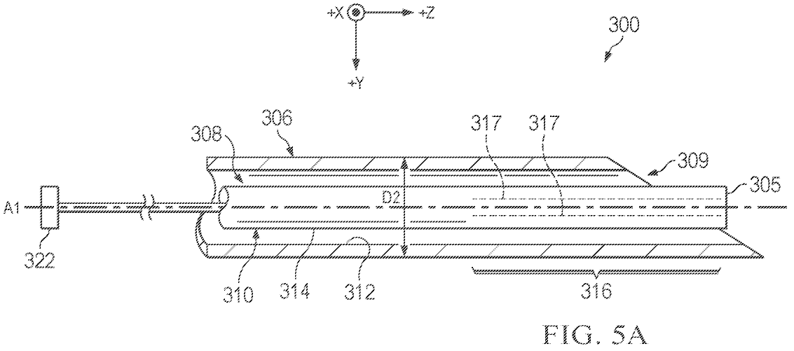

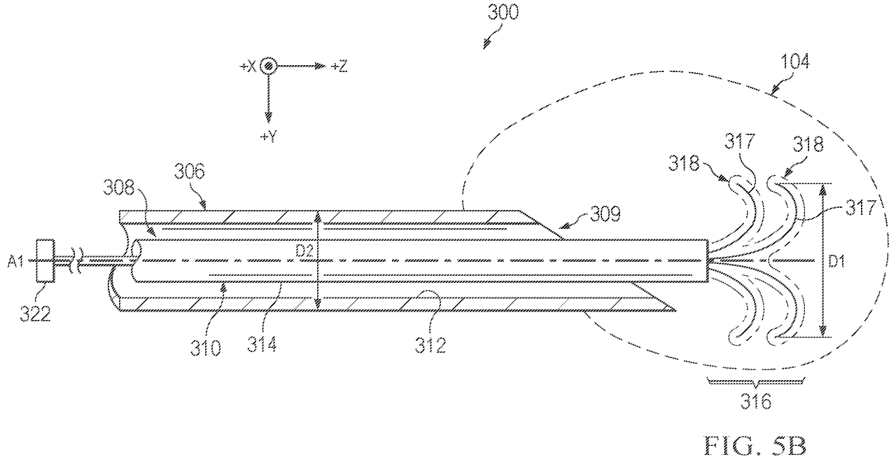

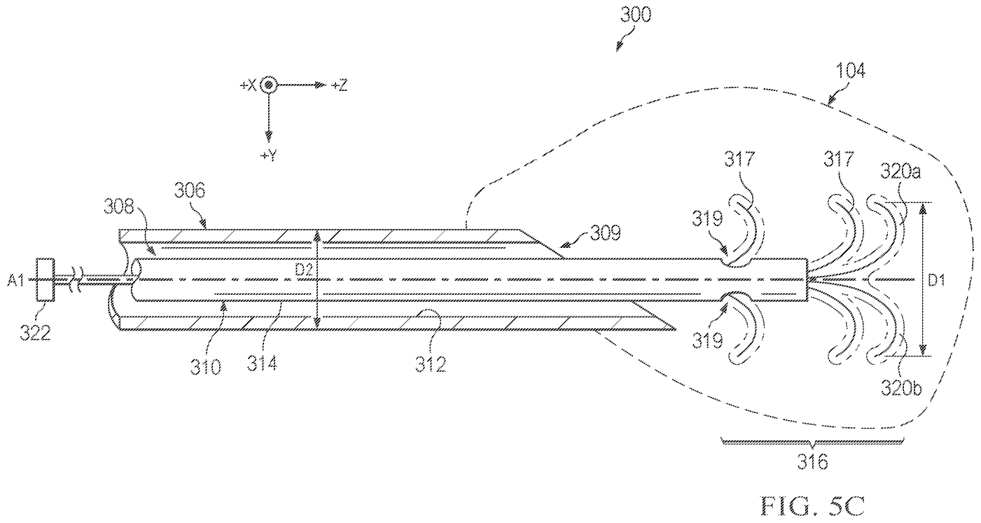

FIGS. 5A-5C illustrate a cross-sectional side view of a medical instrument system 300 including an elongated tool 306 (e.g., the elongated tool 106) in which extends an electrically conductive stylet 310 (e.g., the stylet 110) including a shaft portion 314 and an expandable electrode portion 316. The elongated tool 306 may be flexible and may include an inner wall 312 defining a lumen 308. The lumen 308 may extend to a distal aperture 309. An interior diameter or dimension of the tool 306 may have the width dimension D2. In some examples, the shaft portion may include a tapered distal end 305 that allows the stylet 310 to gain access to the target tissue 104. In this example, the expandable electrode portion 316 may include a plurality of electrode tines 317, and the shaft portion 314 may be cannulated, with the electrode tines 317 compressed and recessed within the shaft portion 314, as shown in FIG. 5A. As shown in the examples of FIG. 5B and 5C, after the shaft portion 314 is extended distally of the aperture 309 and into the target tissue 104, the electrode tines 317 may be extended to expand to a width dimension D1 that is larger than the width dimension D2 of the tool 306. In the examples of FIGS. 5A-C, the electrode tines 317 may each be curved and may flare out from the shaft portion 314 and into the target tissue 104 to create path 318. The electrode tines 317 may each tunnel (e.g., create generally arc-shaped routes) through the target tissue 104. The electrode tines 317 may be formed of a shape-memory material such as nitinol and may have a preset arc shape or may be otherwise pre-bent such that they flare and curl outward from the axis A1 when released from the confines of the shaft portion 314. As shown in the example of FIG. 5C, the electrode tines 317 may also or alternatively extend from apertures or openings 319 along a length of the shaft portion 314 and may curve outward radially from the axis A1 and into the target tissue 104. The path 318 created by the tines 317 may include discrete or discontinuous path segments 320a, 320b that extend away from the central axis A1. The path segments 320a, 320b may extend in different directions relative to the axis A1. For the example, the path segment 320a may initially extend in a generally −Y, +Z direction and then may curve in a generally −Y, −Z direction. The path segment 320b may extend initially in a generally +Y, +Z direction and then may curve in a generally +Y, −Z direction. The path segments may also extend in various +/−X directions. Electricity may be delivered though the expandable electrode portion 316 to the target tissue 104 surrounding the path 318. The expandable electrode portion 316 may allow the stylet to access a larger zone and deliver electricity to a larger region of the target tissue 104 than the tool 306 alone could access in a single deployment.

In some examples, an actuator 322 may be coupled to the expandable electrode portion 316 to cause relative motion between the expandable electrode portion 316 and the shaft portion 314. For example, the actuator 322 may be coupled to the expandable electrode portion 316 such that as the actuator 322 is moved (e.g., displaced longitudinally or rotated), the expandable electrode portion 316 may advance distally relative to the shaft portion 314. The distal advancement of the expandable electrode portion 316 may cause the tines 317 to extend and curve distally of the distal end 307 and/or to extend through the apertures 319. The tines 317 may have a pre-set shape memory or may be otherwise pre-curved such that they arc away from the axis A1 when extended from the shaft portion 314. A reverse motion of the actuator 322 may cause the tines 317 to retract into the shaft portion 314.

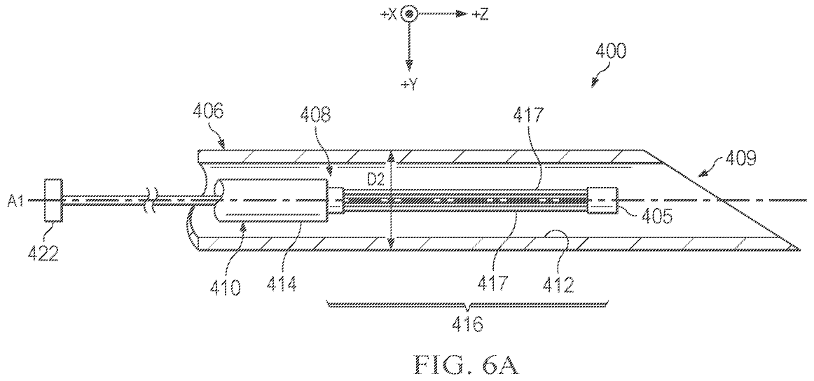

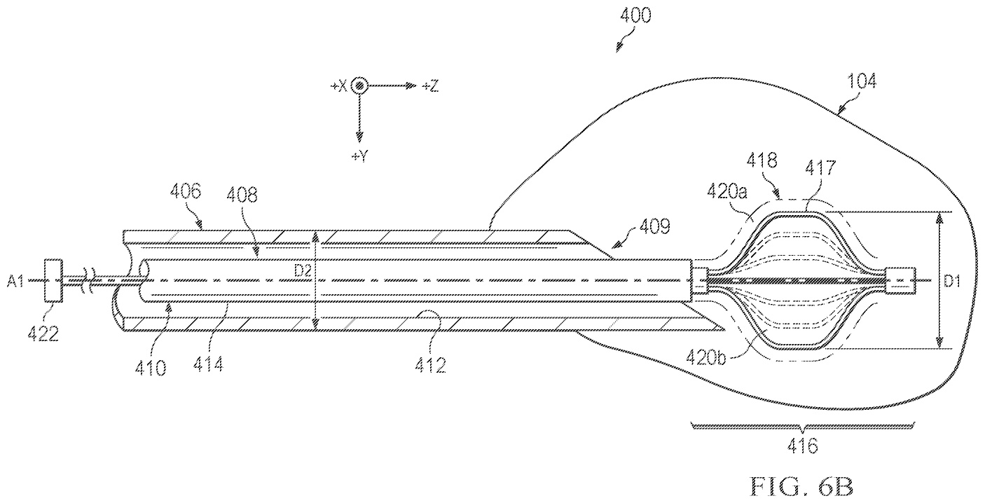

FIGS. 6A-6B illustrate a cross-sectional side view of a medical instrument system 400 including an elongated tool 406 (e.g., the elongated tool 106) in which extends an electrically conductive stylet 410 (e.g., the stylet 110) including a shaft portion 414 and an expandable electrode portion 416. The elongated tool 406 may be flexible and may include an inner wall 412 defining a lumen 408. The lumen 408 may extend to a distal aperture 409. An interior diameter or dimension of the tool 406 may have the width dimension D2. In some examples, the stylet 410 may include a tapered distal end 405 that allows the stylet 410 to pierce or otherwise gain access to the target tissue 104. In this example, the expandable electrode portion 416 may include an expandable basket portion comprising a plurality of electrode splines 417. As shown in FIG. 6A, the electrode splines 417 may be in a retracted, low-profile configuration within the tool 406. As shown in FIG. 6B, after the expandable portion 416 is extended distally of the aperture 409 and into the target tissue 104, the electrode splines 417 may be extended to expand to a width dimension D1 that is larger than the width dimension D2 of the tool 406. The electrode splines 417 may each arch outward away from the axis A1 and into the target tissue 104 to slice a radial path 418. In this example (or other examples describe herein), the expandable electrode portion, including the splines 417, may be expanded while a high frequency cutting current is applied to the expandable portion. The cutting current may facilitate or intensify the slicing action so that less mechanical force may be needed. The current applied for cutting may have a different waveform than a current that may be subsequently used for tissue ablation using the same expandable electrode portion. In some examples, the expandable electrode portion 416 may be formed from a nitinol tube with slits cut along the longitudinal dimension of the tube to create the splines 417 in the remaining tube. In other examples, the splines may include an array of bundled wires. The path 418 created by the splines 417 may include discrete or discontinuous path segments 420a, 420b that extend away from the central axis A1. The path segments 420a, 420b may extend in different directions relative to the axis A1. For the example, the path segment 420a may extend in a generally −Y direction. The path segment 420b may extend initially in a generally +Y direction. The path segments may also extend in various +/−X directions. Electricity may be delivered though the expandable electrode portion 416 to the target tissue 104 surrounding the path 418. The expandable electrode portion 416 may allow the stylet to access a larger zone and deliver electricity to a larger region of the target tissue 104 than the tool 406 alone could access in a single deployment.

In some examples, an actuator 422 may be coupled to the expandable electrode portion 416 and extend through the shaft portion 414 to cause expansion of the expandable electrode portion 416. For example, the actuator 422 may be coupled to the expandable electrode portion 416 such that as the actuator 422 is moved (e.g., displaced longitudinally or rotated), the expandable electrode portion 416 may longitudinally compress, causing the splines 417 to arch outward and create radial planar slices in the target tissue 104. In greater detail, the expandable electrode portion 416 may include a distal section 424 and a proximal section 426, and the compression of the expandable electrode portion 416 includes moving the actuator 422 to move the distal section 424 proximally toward the proximal section 426 or to move the proximal section 426 distally toward the distal section 424. A reverse motion of the actuator 422 may cause the splines 417 to retract toward the axis A1.

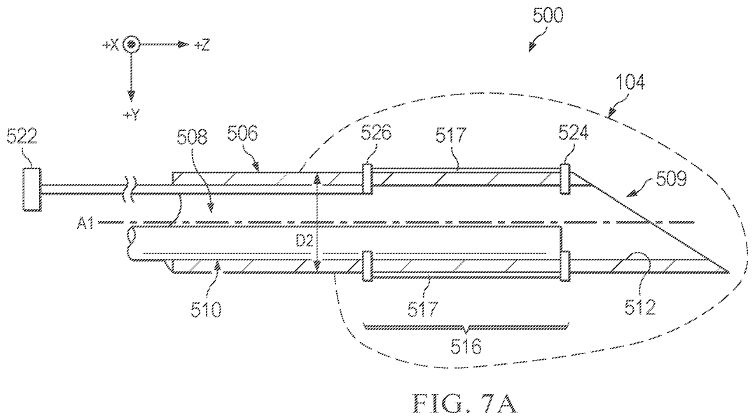

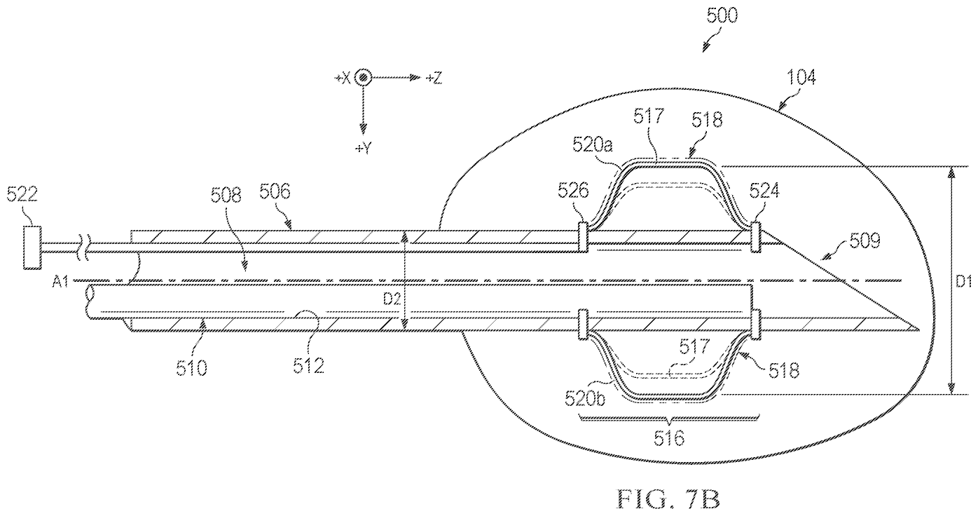

FIGS. 7A-7B illustrate a cross-sectional side view of a medical instrument system 500 including an elongated tool 506 (e.g., the elongated tool 106) in which extends an electrically conductive stylet 510 (e.g., the stylet 110). The elongated tool 506 may be flexible and may include an inner wall 512 defining a lumen 508. The lumen 508 may extend to a distal aperture 509. The stylet 510 may contact the inner wall 512 at one or more points of contact to deliver energy to the tool 506. Various examples for promoting contact between the electrically conductive stylet and the elongated tool are describe in greater detail in U.S. Provisional Patent Application [P06622-US-PRV filed October X, 2022, which is incorporated by reference herein in its entirety.

An interior diameter or dimension of the tool 506 may have the width dimension D2. In this example, the elongated tool 506 may include an expandable electrode portion 516 that comprises a plurality of electrode splines 517. As shown in FIG. 7A, the electrode splines 517 may have a retracted, low-profile configuration as the tool 506 is introduced into the target tissue 104. As shown in FIG. 7B, after the expandable portion 516 is extended into the target tissue 104, the electrode tines 517 may be extended to expand to a width dimension D1 that is larger than the width dimension D2 of the tool 506. The electrode splines 517 may each arch outward away from the axis A1 and into the target tissue 104 to slice radial path 518. In some examples, the expandable electrode portion 516 may be formed from a nitinol tube with slits cut along the longitudinal dimension of the tube to create the splines 517 in the remaining tube. In other examples, the splines may include an array of bundled wires. The path 518 created by the splines 517 may include discrete or discontinuous path segments 520a, 520b that extend away from the central axis A1. The path segments 520a, 520b may extend in different directions relative to the axis A1. For the example, the path segment 520a may extend in a generally −Y direction. The path segment 520b may extend initially in a generally +Y direction. The path segments may also extend in various +/−X directions. Electricity may be delivered though the stylet 510 to the tool 506 and the expandable electrode portion 516. The expandable electrode portion 516, in contact with the target tissue 104, delivers the energy to the tissue surrounding the path 518. The expandable electrode portion 516 may allow the tool to access a larger zone and deliver electricity to a larger region of the target tissue 104 than the unexpanded tool 506 could access in a single deployment.

In some examples, an actuator 522 may be coupled to the expandable electrode portion 516 to cause expansion of the expandable electrode portion 516. For example, the actuator 522 may be coupled to the expandable electrode portion 516 such that as the actuator 522 is moved (e.g., displaced longitudinally or rotated), the expandable electrode portion 516 may longitudinally compress, causing the splines 517 to arch outward and create radial planar slices in the target tissue 104. In greater detail, the expandable electrode portion 516 may include a distal section 524 and a proximal section 526, and the compression of the expandable electrode portion 516 includes moving the actuator 522 to move the distal section 524 proximally toward the proximal section 526 or to move the proximal section 526 distally toward the distal section 524. A reverse motion of the actuator 522 may cause the splines 517 to retract toward the axis A1.

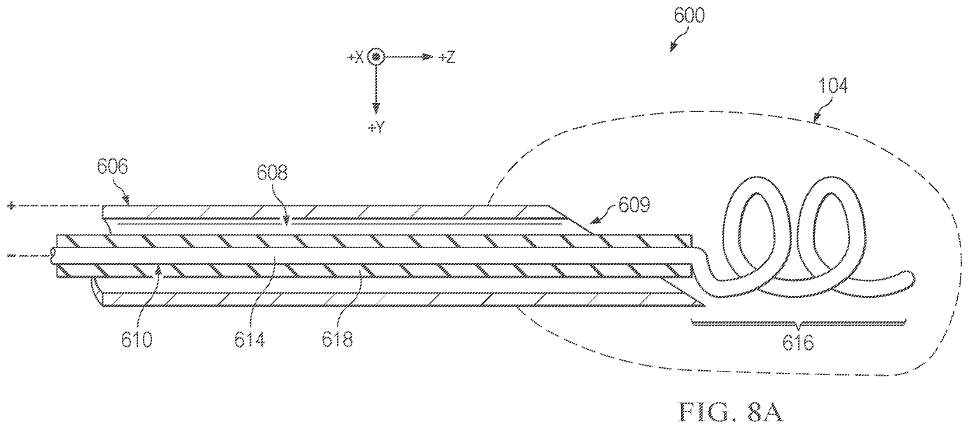

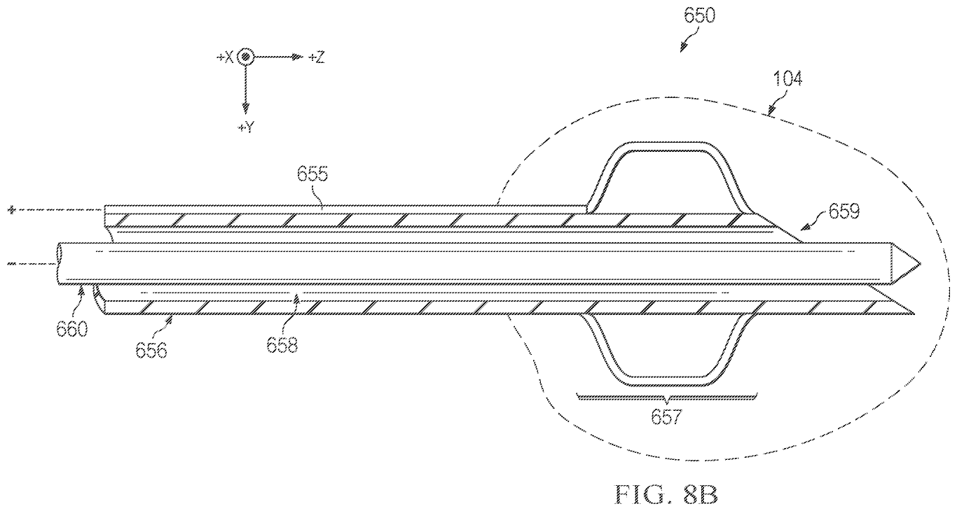

The examples described in FIGS. 1-7B may be monopolar energy delivery assemblies with a grounding electrode placed apart from the electrically conductive tool, on or in the patient anatomy. Alternatively, the examples may be configured as bipolar energy delivery assemblies. FIG. 8A illustrates a cross-sectional side view of a medical instrument system 600 with a bipolar energy delivery assembly including an electrically conductive elongated tool 606 (e.g., the elongated tool 106) in which extends an electrically conductive stylet 610 (e.g., the stylet 110), including a shaft portion 614 and an expandable electrode portion 616. In this example, an insulation member 618 extends around at least a part of the shaft portion 614. The insulation member 618 may be, for example, an insulating sleeve that of extends around the shaft portion 614 to prevent electricity from the shaft portion 614 from flowing directly to the tool 606 along the length of the shaft portion. In operation, this bi-polar assembly may direct energy (e.g., from a generator 124) along the shaft portion 614 of the stylet 610 to the expandable electrode portion 616 and into the target tissue 104. In this example, the electrically conductive tool 606 is a ground electrode through which the energy from the expandable electrode portion 616 and the target tissue 104 may be directed to an electrical ground. The expandable electrode portion 616 may be any of the expandable electrode portions discussed herein.

FIG. 8A illustrates a cross-sectional side view of a medical instrument system 600 with a bipolar energy delivery assembly including an electrically conductive elongated tool 606 (e.g., the elongated tool 106) through which a lumen 608 may extend to a distal aperture 609. An electrically conductive stylet 610 (e.g., the stylet 110) may extend within the lumen 608. The stylet 610 may include a shaft portion 614 and an expandable electrode portion 616. In this example, an insulation member 618 extends around at least a part of the shaft portion 614. The insulation member 618 may be, for example, an insulating sleeve that of extends around the shaft portion 614 to prevent electrical current from the shaft portion 614 from flowing directly to the tool 606 along the length of the shaft portion. In operation, this bi-polar assembly may direct energy (e.g., from a generator 124) along the shaft portion 614 of the stylet 610 to the expandable electrode portion 616 and into the target tissue 104. In this example, the electrically conductive tool 606 is a ground electrode. Electrical current from the expandable electrode portion 616 may follow a path or route through the target tissue 104 to the grounded tool 606. The distance between the active area of the electrode portion 616 and the tool 606 may influence the size of the ablation region. For example, a larger distance may result in a larger ablation area than a smaller distance. Optionally, a distal portion of the electrode portion 616 may be insulated to direct the electrical current into the target tissue 104 in a predictable direction.

FIG. 8B illustrates a cross-sectional side view of a medical instrument system 650 with a bipolar energy delivery assembly including an elongated tool 656 (e.g., the elongated tool 106) through which a lumen 658 may extend to a distal aperture 659. In this example, the tool 656 may be formed of or insulated with an electrical insulation material (e.g., a polyester, polymer, or ceramic insulation material). In this example, an expandable electrode portion 657 may be coupled to the elongated tool 656. The expandable electrode portion 657 may be in electrical connection with an electrical ground via wires or other circuitry 655 that extends along an outer surface of the tool 656. The expandable electrode portion 657 may include, for example, an electrically conductive expandable basket as described in FIG. 7A/7B or an inflatable balloon with one or more attached electrodes. An electrically conductive stylet 660 (e.g., the stylet 110) may extend within the lumen 658 and may be in electrical connection with an electrical source (e.g., generator 124). In operation, the tool 656 may be extended into and expanded within the target tissue 104. The stylet 660 may be extended distally of the aperture 659 and into the target tissue 104. The bi-polar assembly may direct energy (e.g., from a generator 124) along the stylet 660 into the target tissue 104. In this example, the expandable electrode portion 657 may serve as a ground electrode, directing current from the target tissue 104 through the expandable electrode portion 657 to the circuitry 655 of the stylet 610 toward the electrical ground. Thus, electrical current from the stylet 660 may follow a path or route through the target tissue 104 to the grounded expandable electrode portion 657. The distance between the active area of the expandable electrode portion 657 and the stylet 660 may influence the size of the ablation region. For example, a larger distance may result in a larger ablation area than a smaller distance.

In some bi-polar examples with tines (or splines) as in FIGS. 3A-7B, some tines may be different polarities from others of the tines (or splines) such that some tines serve as active electrodes and some serve as ground electrodes.

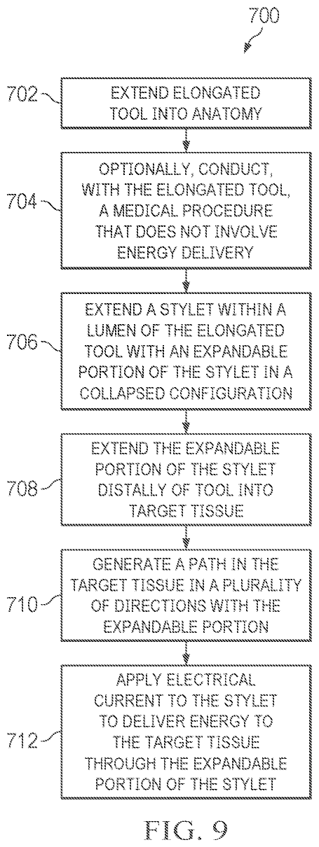

FIG. 9 is a flowchart illustrating a method 700 for delivering energy to a target tissue. The methods described herein are illustrated as a set of operations or processes and are de scribed with continuing reference to additional figures. Not all of the illustrated processes may be performed in all embodiments of the methods. Additionally, one or more processes that are not expressly illustrated in may be included before, after, in between, or as part of the illustrated processes. In some embodiments, one or more of the illustrated processes may be omitted. In some embodiments, one or more of the processes may be implemented, at least in part, in the form of executable code stored on non-transitory, tangible, machine-readable media that when run by one or more processors (e.g., the processing units of a control system such as control system 812) may cause the one or more processors to perform one or more of the processes. In one or more embodiments, the processes may be performed by a control system.

At a process 702, an elongated tool may be extended into a patient anatomy. For example, as shown in FIG. 1, the elongated tool 106 may be a needle extended into the anatomic passageway 102 of a patient and into the target tissue 104. Optionally, the elongated tool may be delivered to a deployment location by a delivery device (e.g., delivery device 111) and the tool may be extended from the delivery device.

At an optional process 704, a medical procedure, such as a biopsy or other procedure that does not involve energy delivery, may be performed with the elongated tool. For example, the elongated tool (e.g., tool 106) may be a cannulated needle that may also be used to perform a biopsy, prior to an energy delivery procedure such as ablation or electroporation. The needle may be delivered into the target tissue and may first be used alone or with a non-energized stylet to sample tissue in a biopsy procedure. If the biopsy procedure confirms that the target tissue is diseased or otherwise may benefit from energy therapy, the processes 706-712 may be conducted to treat the tissue. For example, an electrode stylet may be inserted through the needle lumen and into the target tissue. The current from the electrode stylet may provide a means to treat the target tissue as described. Thus, the cannulated needle may serve as a tool to reach target tissue to conduct multiple procedures, including a biopsy procedure in which the lumen of the needle is used to receive tissue during a biopsy and an energy delivery procedure in which the wall surrounding the lumen of the needle provides a surface to create electrical contact points with the electrode stylet during an electroporation or ablation procedure. Using the same tool for biopsy and for energy therapy may allow for a more efficient and shorter duration medical treatment, without the need to deploy multiple tools.

At a process 706, a conductive stylet may be extended within the elongated tool with an expandable electrode portion of the stylet in a collapsed configuration. The “collapsed configuration,” as used herein, refers to a constrained configuration, a low-profile configuration, or other unexpanded configuration. For example, as shown in FIG. 2A, the electrically conductive stylet 160 may be extended into the elongated tool 106 with the expandable electrode portion 166 in the collapsed configuration. In other examples the conductive stylet may include any other stylet configurations described herein. In some examples, the conductive stylet may be within the elongated tool (e.g., in the collapsed configuration) when the elongated tool is extended into the patient anatomy.

At a process 708, the expandable electrode portion may be extended distally of the elongated tool and into target tissue. For example, as shown in FIG. 2B, the expandable electrode portion 166 of the electrically conductive stylet 160 may extend beyond the distal aperture 159 of the tool 156 and into the target tissue 104. In this example the expandable electrode portion may be rotated or twisted into the target tissue 104

At a process 710, the expandable electrode portion 166 may expand within the target tissue 104 to generate a path in the target tissue that may extend in a plurality of directions. For example, as shown in FIG. 2B, the expandable electrode portion 166 may have an expanded diameter greater D1 greater than the collapsed diameter D2 and thus may expand as it exits the constraints of the tool 156. The expandable electrode portion 166 may generate the path 168 by cutting, slicing, tunneling, boring or otherwise penetrating into the target tissue. The path 168 may extend in different directions along path segments. The path segments that form the path may be continuous or discontinuous. For example. the expandable electrode portion 166 may be rotated or twisted to create a helical or twisted path 168 as the expandable electrode portion 166 advances along the axis A1 into the target tissue 104. The continuous path 168 may include path segments 170a, 170b that extend in different directions. For example, the path segment 170a may extend in a generally −Y, +Z direction, the path segment 170b may extend in a generally −Y, −Z direction, the path segment 170c may extend in a generally +Y, −Z, and the path segment 170d may extend in a generally +Y, +Z direction. In other examples, the path created by the expandable electrode portion may include discontinuous or discrete path segments. In some examples, as shown in FIGS. 3A-3C the expandable electrode portion 216 may be positioned along the axis A1 in the target tissue 104 while still in a collapsed configuration and may be expanded from the position along the axis A1 by an actuator 222 to generate the paths in a plurality of directions.

At a process 712, an electrical current may be applied to the stylet and to the expandable electrode portion. For example, in FIG. 2B, an electrical current may be conducted along the shaft portion 164 of the electrically conductive stylet 160 to the expandable electrode portion 166. The electrical current may flow into the tissue 104 to perform an ablation or electroporation of the target tissue. In monopolar examples, a grounding pad or other grounding tool may be placed on the patient anatomy to ground the electric current. In bi-polar examples (e.g., FIGS. 8A and 8B, for example) at least a portion of the elongated tool may be insulated from at least a portion of the stylet and the current may be directed from the target tissue to ground through the elongated tool or electrically conductive circuits coupled thereto.

In some examples, medical procedure may be performed using hand-held or otherwise manually controlled systems and tools of this disclosure. In other examples, the described imaging probes and tools many be manipulated with a robot-assisted medical system as shown in FIG. 10. FIG. 10 illustrates a robot-assisted medical system 800. The robot-assisted medical system 800 generally includes a manipulator assembly 802 for operating a medical instrument system 804 (including, for example, medical instrument system 100 or any of the medical instrument systems described herein) in performing various procedures on a patient P positioned on a table T in a surgical environment 801. The manipulator assembly 802 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. A master assembly 806, which may be inside or outside of the surgical environment 801, generally includes one or more control devices for controlling manipulator assembly 802. Manipulator assembly 802 supports medical instrument system 804 and may include a plurality of actuators or motors that drive inputs on medical instrument system 804 in response to commands from a control system 812. The actuators may include drive systems that when coupled to medical instrument system 804 may advance medical instrument system 804 into a naturally or surgically created anatomic orifice. Other drive systems may move the distal end of medical instrument system 804 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulatable end effector of medical instrument system 804 for grasping tissue in the jaws of a biopsy device and/or the like.

Robot-assisted medical system 800 also includes a display system 810 for displaying an image or representation of the surgical site and medical instrument system 804 generated by a sensor system 808, which may include an endoscopic imaging system. Display system 810 and master assembly 806 may be oriented so operator O can control medical instrument system 804 and master assembly 806 with the perception of telepresence.

In some examples, medical instrument system 804 may include components for use in surgery, biopsy, ablation, illumination, irrigation, or suction. Medical instrument system 804, together with sensor system 808 may be used to gather (i.e., measure) a set of data points corresponding to locations within anatomic passageways of a patient, such as patient P. In some examples, medical instrument system 804 may include components of the endoscopic imaging system, which may include an imaging scope assembly or imaging that records a concurrent or real-time image of a surgical site and provides the image to the operator or operator O through the display system 810. The concurrent image may be, for example, a two or three-dimensional image captured by an imaging instrument positioned within the surgical site. In some examples, the endoscopic imaging system components may be integrally or removably coupled to medical instrument system 804. However, in some examples, a separate endoscope, attached to a separate manipulator assembly may be used with medical instrument system 804 to image the surgical site. The endoscopic imaging system may be implemented as hardware, firmware, software, or a combination thereof which interact with or are otherwise executed by one or more computer processors, which may include the processors of the control system 812.

The sensor system 808 may include a position/location sensor system (e.g., an electromagnetic (EM) sensor system) and/or a shape sensor system for determining the position, orientation, speed, velocity, pose, and/or shape of the medical instrument system 804.

Robot-assisted medical system 800 may also include control system 812. Control system 812 includes at least one memory 816 and at least one computer processor 814 for effecting control between medical instrument system 804, master assembly 806, sensor system 808 (including endoscopic imaging system), intra-operative imaging system 818, and display system 810. Control system 812 also includes programmed instructions (e.g., a non-transitory machine-readable medium storing the instructions) to implement some or all of the methods described in accordance with aspects disclosed herein, including instructions for providing information to display system 810.

Control system 812 may further include a virtual visualization system to provide navigation assistance to operator O when controlling medical instrument system 804 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

FIG. 11A is a simplified diagram of a medical instrument system 900 configured in accordance with various embodiments of the present technology. The medical instrument system 900 includes an elongate flexible device 902 (e.g., delivery device 111), such as a flexible catheter, coupled to a drive unit 904. The elongate flexible device 902 includes a flexible body 916 having a proximal end 917 and a distal end or tip portion 918. The medical instrument system 900 further includes a tracking system 930 for determining the position, orientation, speed, velocity, pose, and/or shape of the distal end 918 and/or of one or more segments 924 along the flexible body 916 using one or more sensors and/or imaging devices as described in further detail below.

The tracking system 930 may optionally track the distal end 918 and/or one or more of the segments 924 using a shape sensor 922. The shape sensor 922 may optionally include an optical fiber aligned with the flexible body 916 (e.g., provided within an interior channel (not shown) or mounted externally). The optical fiber of the shape sensor 922 forms a fiber optic bend sensor for determining the shape of the flexible body 916. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. Pat. No. 7,781,724 (filed Sep. 26, 2006, disclosing “Fiber optic position and shape sensing device and method relating thereto”; U.S. Pat. No. 7,772,541, filed Mar. 12, 2008, titled “ Fiber Optic Position and/or Shape Sensing Based on Rayleigh Scatter”; and U.S. Pat. No. 6,389,187, filed Apr. 21, 2000, disclosing “Optical Fiber Bend Sensor,” which are all incorporated by reference herein in their entireties. In some embodiments, the tracking system 930 may optionally and/or additionally track the distal end 918 using a position sensor system 920. The position sensor system 920 may be a component of an EM sensor system with the position sensor system 920 including one or more conductive coils that may be subjected to an externally generated electromagnetic field. In some embodiments, the position sensor system 920 may be configured and positioned to measure six degrees of freedom (e.g., three position coordinates X, Y, and Z and three orientation angles indicating pitch, yaw, and roll of a base point) or five degrees of freedom (e.g., three position coordinates X, Y, and Z and two orientation angles indicating pitch and yaw of a base point). Further description of a position sensor system is provided in U.S. Pat. No. 6,380,732, filed Aug. 9, 1999, disclosing “Six-Degree of Freedom Tracking System Having a Passive Transponder on the Object Being Tracked,” which is incorporated by reference herein in its entirety. In some embodiments, an optical fiber sensor may be used to measure temperature or force. In some embodiments, a temperature sensor, a force sensor, an impedance sensor, or other types of sensors may be included within the flexible body. In various embodiments, one or more position sensors (e.g. fiber shape sensors, EM sensors, and/or the like) may be integrated within the medical instrument 926 and used to track the position, orientation, speed, velocity, pose, and/or shape of a distal end or portion of medical instrument 926 using the tracking system 930.

The flexible body 916 includes a channel 921 sized and shaped to receive a medical instrument 926 (e.g., elongated tool 106). FIG. 11B, for example, is a simplified diagram of the flexible body 916 with the medical instrument 926 extended according to some embodiments. In some embodiments, the medical instrument 926 may be used for procedures such as imaging, visualization, surgery, biopsy, ablation, illumination, irrigation, and/or suction. The medical instrument 926 can be deployed through the channel 921 of the flexible body 916 and used at a target location within the anatomy. The medical instrument 926 may include, for example, image capture probes, biopsy instruments, ablation needles, electroporation needles, laser ablation fibers, and/or other surgical, diagnostic, or therapeutic tools, including any of the instrument systems described above. The medical instrument 926 may be used with an imaging instrument (e.g., an image capture probe) within the flexible body 916. The imaging instrument may include a cable coupled to the camera for transmitting the captured image data. In some embodiments, the imaging instrument may be a fiber-optic bundle, such as a fiberscope, that couples to an image processing system 931. The imaging instrument may be single or multi-spectral, for example capturing image data in one or more of the visible, infrared, and/or ultraviolet spectrums. The medical instrument 926 may be advanced from the opening of channel 921 to perform the procedure and then be retracted back into the channel 921 when the procedure is complete. The medical instrument 926 may be removed from the proximal end 917 of the flexible body 916 or from another optional instrument port (not shown) along the flexible body 916.

The flexible body 916 may also house cables, linkages, or other steering controls (not shown) that extend between the drive unit 904 and the distal end 918 to controllably bend the distal end 918 as shown, for example, by broken dashed line depictions 919 of the distal end 918. In some embodiments, at least four cables are used to provide independent “up-down” steering to control a pitch of the distal end 918 and “left-right” steering to control a yaw of the distal end 918. Steerable elongate flexible devices are described in detail in U.S. Pat. No. 9,452,276, filed Oct. 14, 2011, disclosing “Catheter with Removable Vision Probe,” and which is incorporated by reference herein in its entirety. In various embodiments, medical instrument 926 may be coupled to drive unit 904 or a separate second drive unit (not shown) and be controllably or robotically bendable using steering controls.

The information from the tracking system 930 may be sent to a navigation system 932 where it is combined with information from the image processing system 931 and/or the preoperatively obtained models to provide the operator with real-time position information. In some embodiments, the real-time position information may be displayed on the display system 810 of FIG. 10 for use in the control of the medical instrument system 900. In some embodiments, the control system 812 of FIG. 10 may utilize the position information as feedback for positioning the medical instrument system 900. Various systems for using fiber optic sensors to register and display a surgical instrument with surgical images are provided in U.S. Pat. No. 8,900,131, filed May 13, 2011, disclosing “Medical System Providing Dynamic Registration of a Model of an Anatomic Structure for Image-Guided Surgery,” which is incorporated by reference herein in its entirety.

In some embodiments, the medical instrument system 900 may be teleoperated or robot-assisted within the medical system 800 of FIG. 10. In some embodiments, the manipulator assembly 802 of FIG. 10 may be replaced by direct operator control. In some embodiments, the direct operator control may include various handles and operator interfaces for hand-held operation of the instrument.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 812) or may be implemented, at least in part, in the form of executable code stored on non-transitory, tangible, machine-readable media that when run by one or more processors (e.g., the processors 814 of control system 812) may cause the one or more processors to perform one or more of the processes.

One or more elements in examples of this disclosure may be implemented in software to execute on a processor of a computer system such as control processing system. When implemented in software, the elements of the examples of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In one example, the control system supports wireless communication protocols such as Bluetooth, IrDA, HomeRF, IEEE 802.11, DECT, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

The singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. Components described as coupled may be directly or indirectly communicatively coupled. The auxiliary verb “may” likewise implies that a feature, step, operation, element, or component is optional.

While certain exemplary examples of the invention have been described and shown in the accompanying drawings, it is to be understood that such examples are merely illustrative of and not restrictive on the broad invention, and that the examples of the invention not be limited to the specific constructions and arrangements shown and described, since various other modifications may occur to those ordinarily skilled in the art.

1. A system comprising:
an elongated tool through which a lumen extends; and
a stylet including an expandable electrode portion, the expandable electrode portion having a collapsed configuration within the lumen and having an expanded configuration outside of the lumen, a diameter of the expandable electrode portion being larger in the expanded configuration than the collapsed configuration,
wherein in the expanded configuration, the expandable electrode portion is configured to create a plurality of path segments in a target tissue, wherein each path segment in the plurality of path segments is extended in a different direction and
wherein the expandable electrode portion in the expanded configuration is configured to deliver energy to ablate the target tissue.

2. The system of claim 1 wherein the elongated tool and the stylet are flexible.

3. (canceled)

4. The system of claim 1 wherein the expandable electrode portion is helically shaped.

5. The system of claim 1 wherein the expandable electrode portion is expandable to a diameter greater than an outer diameter of the elongated tool when extended distally of the lumen.

6. The system of claim 5 wherein the expandable electrode portion is formed of nitinol.

7. The system of claim 5 wherein the expandable electrode portion has a straightened configuration in the lumen.

8. The system of claim 5 wherein the stylet is rotatable to twist into the target tissue creating the path segments in a plurality of directions.

9. The system of claim 1 wherein the stylet includes a cannulated shaft and wherein the expandable electrode portion includes a plurality of electrode tines that extend from the cannulated shaft.

10. The system of claim 9 wherein the electrode tines are straight and extend outward from the cannulated shaft in a distal direction and the plurality of path segments in the target tissue are generally straight.

11. The system of claim 9 wherein the electrode tines extend from a distal end of the cannulated shaft.

12. The system of claim 9 wherein the electrode tines extend through openings along the cannulated shaft.

13. The system of claim 9 wherein the electrode tines are collapsible into the elongated tool by proximal motion of the stylet.

14. The system of claim 9 wherein the electrode tines are straight and extend outward from the cannulated shaft in a proximal direction and the plurality of path segments in the target tissue are generally straight.

15. The system of claim 9 further comprising an actuator configured to move the electrode tines relative to the cannulated shaft.

16. The system of claim 9 wherein the electrode tines curve away from a central axis of the cannulated shaft and the plurality of path segments in the target tissue are generally curved.

17. The system of claim 1 wherein the stylet includes a cannulated shaft, and the expandable electrode portion includes an expandable basket portion that has a low profile configuration to enter the target tissue and has an expanded profile configuration with a plurality of splines configured to slice the plurality of path segments in the target tissue.

18. The system of claim 17 wherein the plurality of splines is formed from a nitinol tube.

19. The system of claim 17 wherein the plurality of splines includes an array of wires.

20. The system of claim 17 further comprising an actuator movable to transition the expandable basket portion between the collapsed and expanded profile configurations.

21. The system of claim 18 wherein the expandable basket portion includes an active electrode and the shaft is grounded.

22-29. (canceled)
